(19) United States
(12) Patent Application Publication
(10) Pub. No.: US 2025/0195871 A1
(43) Pub. Date: Jun. 19, 2025

(54) CONTROL UNIT FOR A BLOOD PUMP, PUMP SYSTEM, AND METHOD

(71) Applicant: Berlin Heart GmbH, Berlin (DE)

(72) Inventors: Matthias Kiesner, Berlin (DE); Oliver Peters, Berlin (DE); Robert Steingräber, Berlin (DE)

(73) Assignee: Berlin Heart GmbH, Berlin (DE)

(21) Appl. No.: 18/849,096

(22) PCT Filed: Mar. 21, 2023
(86) PCT No.: PCT/EP2023/057218

§ 371 Date: Sep. 20, 2024

(30) Foreign Application Priority Data
Mar. 21, 2022 (EP) 22163251.6

(51) Int. Cl. A61M 60/538; A61M 60/178; A61M 60/232; A61M 60/419; A61M 60/822; G16H 40/63

(52) U.S. Cl. CPC A61M 60/538; A61M 60/178; A61M 60/232; A61M 60/419; A61M 60/822; G16H 40/63; A61M 2205/103; A61M 2205/3317; A61M 2205/332; A61M 2205/3327; A61M 2205/50

(57) ABSTRACT

The application relates to a control unit (200) for a blood pump (300) with a magnetically supported rotor (320) and a stator (340) which is configured to generate a stator magnetic field for exerting a bearing force on the rotor (320) along a bearing direction of action. The blood pump (300) is configured to provide a measurement signal which comprises a disturbance component dependent on a rotation and/or lateral movement of the rotor (320). The control unit (200) is configured to determine, on the basis of the measurement signal, a disturbance signal dependent on the disturbance component, and to determine, on the basis of the measurement signal and the determined disturbance signal, a specification for a bearing control signal in such a way that the rotor (320) is supported in the housing (301) without contact by means of the bearing force, wherein a power consumed for exerting the bearing force is reduced compared with a specification for the bearing control signal which may be determined without taking the disturbance signal into account. The application also relates to a pump system (100) and to a method for controlling a blood pump (200).

The application relates to a control unit for a blood pump, in particular a rotary fluid pump for cardiac support, a pump system comprising such a control unit and a method for controlling a blood pump.

A blood pump according to the application comprises a rotor, which is magnetically supported in a housing and is rotatable about a rotation axis to convey a fluid, and a stator. The stator is configured to generate a variable stator magnetic field for exerting a variable bearing force on the rotor along a bearing direction of action in accordance with a bearing control signal. The blood pump is configured to provide a measurement signal dependent on a bearing position of the rotor along the bearing direction of action.

Blood pumps of this type as well as corresponding control units and control methods are known from the prior art.

When controlling such a blood pump, it is possible to determine a specification for the bearing control signal on the basis of the measurement signal in such a way that the rotor is supported in the housing without contact along the bearing direction of action by means of the bearing force, which is also referred to below as active magnetic support along the bearing direction of action.

In addition to the bearing position, the measurement signal may also depend on other influences, in particular disturbances. These may originate, for example, from the detection of the measurement signal, such as by means of a position sensor. The bearing control signal determined on the basis of this measurement signal may then include an undesirable component that is not required for the rotor bearing and that may even impair it. This undesirable component may increase the applied bearing force and the associated power consumption; the pumped blood may also be exposed to increased heating.

Against the background of the prior art, the application addresses the problem of providing a control unit for a blood pump, a pump system and a method for controlling a blood pump which avoid or reduce the disadvantages mentioned.

To solve the problem, a control unit according to claim 1, a pump system according to claim 15 and a method according to claim 19 are proposed. Advantageous embodiments and further developments are described in conjunction with the features of the dependent claims.

The proposed control unit is configured to control a blood pump of the above-mentioned type. The blood pump is preferably configured to convey the fluid, in particular blood, from an inlet to an outlet. The inlet and outlet may each be connected directly or indirectly, in particular by means of a cannula, to a heart and/or a blood vessel. The blood pump may be used in particular as a VAD (ventricular assist device).

The blood pump is configured to provide a measurement signal which is dependent on a bearing position of the rotor along the bearing direction of action and which comprises a disturbance component dependent on a rotation of the rotor about the rotation axis and/or a lateral movement of the rotor perpendicular to the bearing direction of action. The measurement signal may be a one- or multi-dimensional measurement signal, for example, it may comprise a single sensor signal or a plurality of similar or different sensor signals and/or signals derived from them. The term “dependent on a lateral movement of the rotor perpendicular to the bearing direction of action” also includes here a dependency on a tilting of the rotor about an axis perpendicular to the rotation axis X.

A disturbance component of the type mentioned may occur, for example, due to measurement errors when detecting or providing the measurement signal and/or due to properties of a sensor arrangement configured to detect the measurement signal. In particular, the disturbance component may be caused by the fact that such a sensor arrangement also detects the rotation and/or lateral movement of the rotor in addition to the bearing position of the rotor along the bearing direction of action. For example, the sensor arrangement may comprise a sensor target arranged eccentrically in or on the rotor in relation to the rotation axis and a sensor component arranged fixed in relation to the stator, wherein the measurement signal corresponds to a distance of the sensor target from the sensor component. The distance may then depend both on the bearing position and on the rotation and/or lateral movement of the rotor, which means that the corresponding measurement signal has a disturbance component of the type mentioned.

The control unit is configured to detect the measurement signal and, on the basis of the measurement signal detected in a detection interval, to determine a disturbance signal dependent on the disturbance component, and to determine, on the basis of the measurement signal and the determined disturbance signal, a specification for the bearing control signal in such a way that the rotor is supported in the housing without contact, in particular along the bearing direction of action, by means of the bearing force, wherein a power consumed for exerting the bearing force is reduced compared with a specification for the bearing control signal which may be determined without taking the disturbance signal into account.

In particular, this means that the power consumed to exert the bearing force when the stator is controlled in accordance with the bearing control signal corresponding to the specification determined taking the disturbance signal into account is lower than a power that is consumed when the stator is controlled in accordance with a bearing control signal that corresponds to a specification that may be determined on the basis of the measurement signal but without determining or taking into account the disturbance signal. By determining and taking into account the disturbance signal, the power consumption and associated effects, such as heating of the pumped blood, may be reduced. This reduction in power consumption may be achieved in particular by reducing the bearing force to be applied, which results from the fact that an effect of the disturbance component dependent on the rotation and/or lateral movement of the rotor is reduced or compensated for.

The specification for the bearing signal determined on the basis of the measurement signal and the determined disturbance signal is also referred to as a disturbance-compensated specification; a specification for the bearing control signal that may be determined without taking the disturbance signal into account is accordingly also referred to as a non-disturbance-compensated specification. Accordingly, the power consumed to exert the bearing force is reduced when the rotor is supported in the housing with the disturbance-compensated specification compared to when the rotor is supported in the housing with a non-disturbance-compensated specification for the bearing control signal.

The control unit is also configured to control the stator according to the bearing control signal in accordance with the determined specification, wherein the control takes place after the detection interval. A delay interval may be provided between the end of the detection interval and the control of the stator. The control unit is preferably configured to carry out the detection of the measurement signal, the determination of the disturbance signal, the determination of the specification for the bearing control signal and the corresponding actuation of the stator several times repeatedly in a sequence of control cycles. The detection interval and/or the delay interval of a given control cycle may at least partially overlap the detection interval and/or the delay interval of a subsequent further control cycle.

Because the disturbance signal is determined on the basis of the measurement signal detected in the detection interval and is taken into account when determining the specification for the bearing control signal, and because the actuation takes place after the detection interval, the control is based on a time characteristic of the measurement signal—and thus of the disturbance component—that precedes the time of control. The reduction in power consumption is therefore made possible by taking into account the previous time curve of the measurement signal under the assumption that the disturbance component at the time of control substantially corresponds to the disturbance component at a previous time. Under this assumption, compensation for the disturbance component (disturbance compensation) may be carried out by correcting the measurement signal and/or the specification for the bearing control signal in accordance with the disturbance signal.

Due to this compensation, a particularly compact and robust design of the blood pump itself may also be possible, as smaller sensors with compromises may be used, or “sensorless” generation of the measurement signal, in which the motor coil windings of the stator act as sensors, is also possible. Changes in the measurement signal due to damage, aging or drift of blood pump components may also be compensated for, which may also increase the service life and reliability of the blood pump. Furthermore, the number of wires that must be provided in a driveline for transmitting the measurement signal is particularly low when the measurement signal is generated without sensors, which may also enable a robust and compact design and/or simplified provision of redundancy.

A time course of the disturbance component may be substantially periodic with a period of rotation of the rotor, hereinafter also referred to as the rotation period. The rotation period may correspond here to the mechanical rotation period or, if a motor with more than one magnetic pole pair in the rotor is used for the drive, also to the electrical rotation period of the magnetic field generated by the stator. The time course of the disturbance component may comprise a substantially periodic component and a component that varies slowly in relation to the rotation period (such as a variable offset). The disturbance signal may also depend on the rotation period, i.e. may be speed-dependent.

In the case of a periodicity with the rotation period, the disturbance component at each time of control substantially corresponds to the disturbance component at a previous time that is one or more rotation periods in the past.

Accordingly, a time course of the determined disturbance signal may be substantially periodic with the rotation period. The time course of the disturbance signal may comprise a substantially periodic component and a component that varies slowly in relation to the rotation period (such as a variable offset). The disturbance signal may be determined under the boundary condition that it is substantially periodic with the rotation period or comprises a substantially periodic component.

The periodicity of the disturbance component may thus be utilized to achieve a reliable reduction in power consumption.

It may be the case that the disturbance component and/or the disturbance signal does not depend on the bearing position of the rotor averaged over a period of rotation of the rotor along the bearing direction of action.

The detection interval may comprise at least one, for example a plurality of, rotation periods, which makes it possible to determine the disturbance signal.

The power consumed to exert the bearing force, which is reduced compared to a specification for the bearing control signal that may be determined without taking the disturbance signal into account, may be a time-averaged power, in particular averaged over at least one period of the rotation of the rotor. An advantageous reduction in this power consumption may occur even if the bearing force to be applied is not reduced at all times (and is possibly even increased at some times).

The compensation of the disturbance component on the basis of the specific disturbance signal may intervene at various points, as explained below.

On the one hand, the control unit may be configured to determine the bearing control signal as a corrected bearing control signal, wherein the determined disturbance signal is applied as a compensation signal to an uncorrected bearing control signal determined on the basis of the measurement signal, so that when the stator is controlled in accordance with the corrected bearing control signal, the bearing force exerted on the rotor is influenced less by the disturbance component of the measurement signal than when the stator is controlled in accordance with the uncorrected bearing control signal.

On the other hand, it may—alternatively or additionally—be provided that the measurement signal is an uncorrected measurement signal and the control unit is configured to determine a corrected measurement signal by applying the determined disturbance signal as a compensation signal to the measurement signal when determining the bearing control signal, so that a disturbance component of the corrected measurement signal which depends on the rotation of the rotor about the rotation axis or the lateral movement of the rotor perpendicular to the rotation axis is less than the disturbance component of the uncorrected measurement signal.

The rotor is actively magnetically supported in the housing along the bearing direction of action by means of the bearing force that may be varied according to the bearing control signal. The rotor may also be supported in the housing in other directions by means of active magnetic bearings and/or in other ways, such as mechanical, hydrodynamic, hydrostatic and/or passive magnetic bearings.

The stator and the rotor may form an unstable controlled system with regard to control of the bearing position of the rotor along the bearing direction of action. For example, the position of the rotor along the bearing direction of action may be destabilized by a passive magnetic support of the rotor in a lateral or radial direction perpendicular to the bearing direction of action. The control unit may be configured to stabilize the unstable controlled system by means of a bearing control.

The control unit may be configured to determine the disturbance signal by means of a learning control, in particular an iteratively learning control and/or a repetitive control and/or a run-to-run control. The disturbance signal may be reliably determined by means of a learning control, especially if the disturbance component is periodic or has a periodic component; furthermore, a slow change in the disturbance component compared to the rotation period may also be reliably compensated for in this way. Various methods of learning control are described, for example, in: Youqing Wang, Furong Gao, Francis J. Doyle, Survey on iterative learning control, repetitive control, and run-to-run control, Journal of Process Control, Volume 19, Issue 10, 2009, Pages 1589-1600, ISSN 0959-1524, doi: 10.1016/j.jprocont.2009.09.006.

Learning control may be implemented in various ways, for example with regard to manipulated and controlled variables. It may be provided that a controlled variable of the learning control is a target bearing force and a manipulated variable of the learning control is the bearing position of the rotor. It may be provided that a controlled variable of the learning control is a target bearing force and a manipulated variable of the learning control is the bearing force exerted on the rotor. It may be provided that a controlled variable of the learning control is a control error of the above-mentioned bearing control and a manipulated variable of the learning control is the bearing position of the rotor along the bearing direction of action.

The learning control may be configured to determine a start and/or end time of a period of rotation of the rotor on the basis of a rotation angle of the rotor about the rotation axis. The control unit may be configured to store a course of the disturbance signal as a function of a rotation angle of the rotor about the rotation point. In this way, good control results may be achieved even if the duration of the rotation period (period duration) varies over time.

The control unit may be configured to determine the bearing control signal in such a way that the rotor is adjusted into a target bearing position at which external forces acting on the rotor along the bearing direction of action add up to a predetermined force, in particular a zero force, and/or at which a power applied to generate the variable stator magnetic field is minimal.

The control unit may be configured to determine a difference between a first power consumption corresponding to the power consumed for exerting the bearing force when using the specification for the bearing control signal determined on the basis of the measurement signal and the disturbance signal (i.e. the disturbance-compensated specification), and a second power consumption corresponding to the power consumed for exerting the bearing force when using the specification for the bearing control signal that may be determined without taking the disturbance signal into account (i.e. the non-disturbance-compensated specification). The control unit may be configured to determine a difference between the first power consumption and a third power consumption corresponding to the power consumed to exert the bearing force when using the specification for the bearing control signal determined in a previous control cycle (i.e. a disturbance-compensated specification of a previous control cycle).

If the first power consumption is greater than the second and/or third power consumption, the control unit may then also be configured to control the stator according to the bearing control signal in accordance with the specification that may be determined without taking the disturbance signal into account (i.e. the non-disturbance-compensated specification) or according to the bearing control signal in accordance with the specification determined in a previous control cycle. In particular, the control unit may be configured to choose between the disturbance-compensated specification and the non-disturbance-compensated specification and/or the disturbance-compensated specification of a previous control cycle, depending on a certain difference. This allows the actual power consumption of the blood pump to be optimized even if the fault-compensated specification (of the current control cycle) temporarily does not lead to a reduction in power consumption.

The preceding control cycle mentioned above may be the immediately preceding control cycle or a control cycle several control cycles in the past. The control unit may be configured to store the specification for the bearing control signal determined in a given control cycle, so that it may be used to control the stator at a later time, in particular if the first power consumption is greater than the second and/or third power consumption. The control unit may be configured accordingly to keep the stored specification ready for access for one or more control cycles.

The proposed pump system comprises
a blood pump and a control unit of the type proposed above, wherein the control unit is configured to control the blood pump,
wherein the blood pump comprises a rotor magnetically supported in a housing and rotatable about a rotation axis for conveying a fluid, and a stator, wherein the stator is configured to generate a variable stator magnetic field for exerting a bearing force on the rotor which may be varied along a bearing direction of action in accordance with a bearing control signal;
wherein the blood pump is configured to provide a measurement signal which is dependent on a bearing position of the rotor along the bearing direction of action and which comprises a disturbance component dependent on a rotation of the rotor about the rotation axis and/or a lateral movement of the rotor perpendicular to the bearing direction of action.

In the proposed pump system, the proposed control unit provides its advantages described above.

The rotor may comprise a sensor target arranged eccentrically in relation to the rotation axis. The blood pump may be configured to provide the measurement signal on the basis of a position of the sensor target. As presented further above, a sensor arrangement with an eccentric sensor target may generate a disturbance component of the type mentioned. By means of the proposed control unit, this disturbance may be compensated for, and thus, for example, a design adjustment towards an axially arranged sensor target or one that coincides with the rotation axis may be avoided.

The measurement signal may correspond to a back-electromotive force (back-EMF) induced by a rotation of the rotor. The measurement signal corresponding to the back-EMF may be detected without an additional sensor by means of the motor coil windings of the stator, which therefore enables “sensorless” generation of the measurement signal.

The blood pump may alternatively or additionally comprise a sensor, for example an eddy current sensor and/or a magnetic field sensor, in particular a Hall sensor, to provide the measurement signal.

The bearing direction of action may be arranged substantially or approximately parallel to the rotation axis. The bearing direction of action may be arranged in a different direction, such as a radial or lateral direction perpendicular to the rotation axis or a direction at a different angle to the rotation axis. The proposed control unit may therefore be used with different types of pumps and bearings.

The proposed method is intended for controlling a blood pump,
wherein the blood pump comprises a rotor magnetically supported in a housing and rotatable about a rotation axis for conveying a fluid, and a stator, wherein the stator is configured to generate a variable stator magnetic field for exerting a bearing force on the rotor which may be varied along a bearing direction of action in accordance with a bearing control signal;
wherein the blood pump is configured to provide a measurement signal which is dependent on a bearing position of the rotor along the bearing direction of action and which comprises a disturbance component dependent on a rotation of the rotor about the rotation axis and/or a lateral movement of the rotor perpendicular to the bearing direction of action.

The method comprises:
detecting the measurement signal,
determining a disturbance signal, dependent on the disturbance component, on the basis of the measurement signal detected in a detection interval,
determining, on the basis of the measurement signal and the determined disturbance signal, a specification for the bearing control signal in such a way that the rotor is supported in the housing without contact by means of the bearing force, wherein a power consumed for exerting the bearing force is reduced compared with a specification for the bearing control signal which may be determined without taking the disturbance signal into account, and
after the detection interval, controlling the stator according to the bearing control signal in accordance with the determined specification.

In particular, the proposed method may be carried out using a control unit of the type proposed above and/or a pump system of the type proposed above and may be further embodied in accordance with the optional features of the control unit and the pump system.

Exemplary embodiments of the subject matter of the application are explained below with reference to drawings. The drawings show:

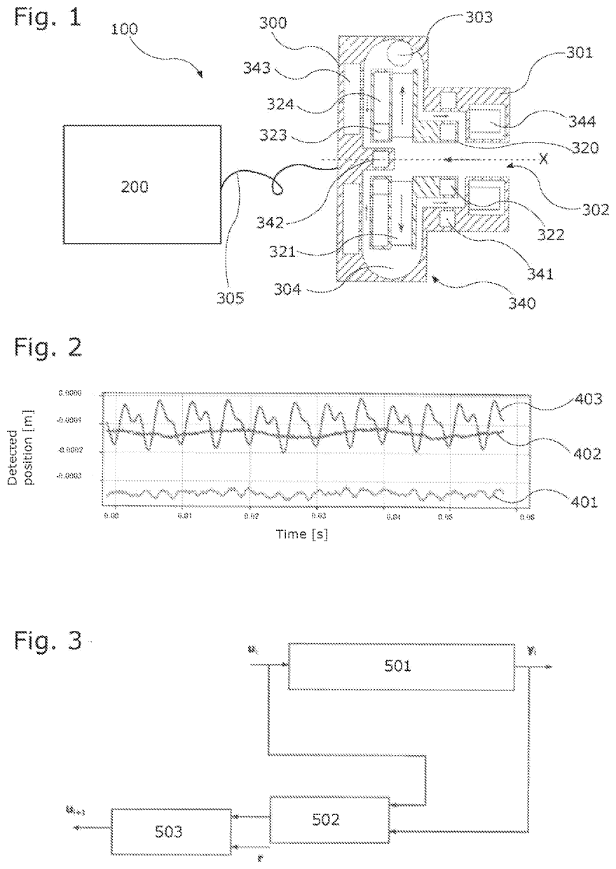

FIG. 1 a schematic representation of a pump system with a blood pump and a control unit,

FIG. 2 a time course of various position signals,

FIG. 3 a schematic representation of a pump control,

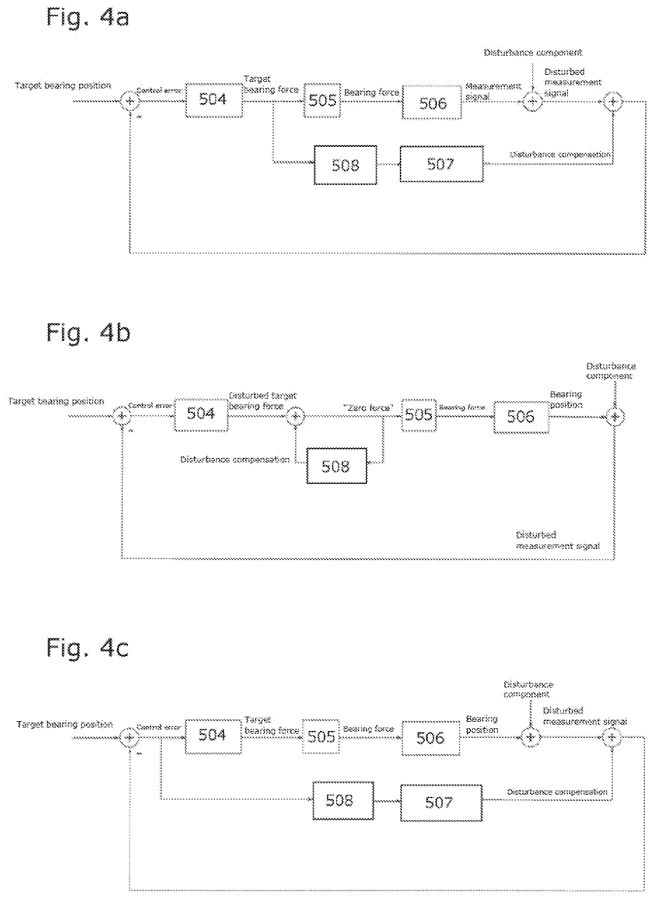

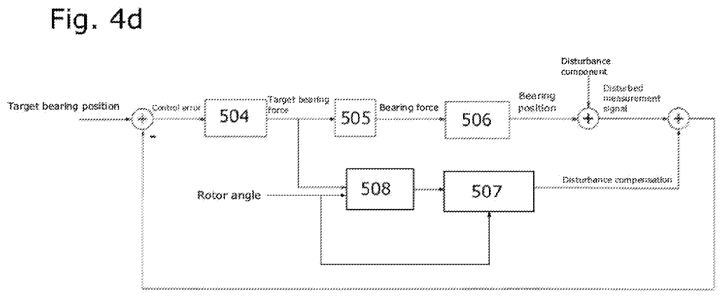

FIGS. 4a to 4d schematic representations of pump controls according to various examples,

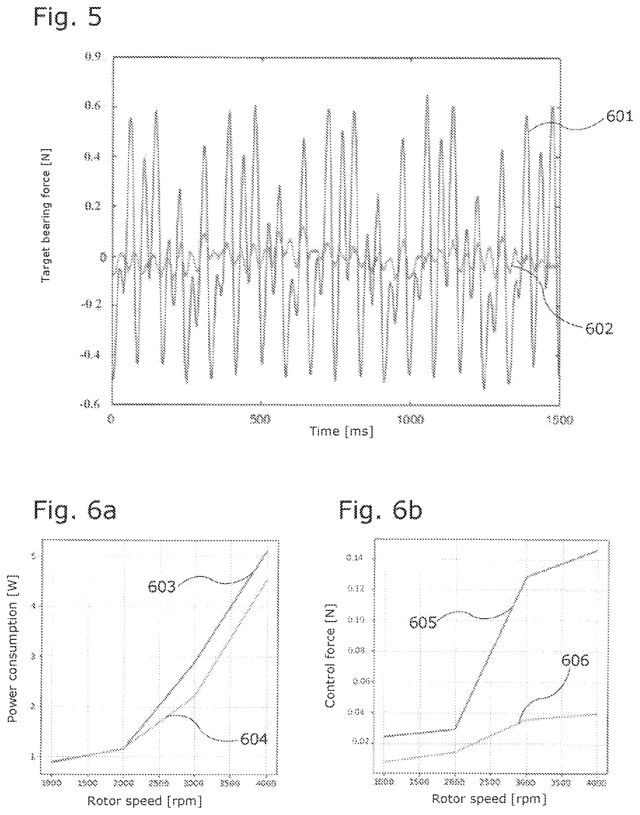

FIG. 5 a time course of a target force on the basis of various specifications for a bearing control signal,

FIG. 6a a power consumption as a function of a rotor speed on the basis of various specifications for the bearing control signal,

FIG. 6b a control force as a function of the rotor speed on the basis of various specifications for the bearing control signal.

Recurring and similar features of different embodiments are provided with identical alphanumeric reference signs in the illustrations.

The pump system 100 shown in FIG. 1 comprises a blood pump 300 and a control unit 200, wherein the control unit 200 is configured to control the blood pump 300.

The blood pump 300 is formed as a rotary fluid pump for cardiac support and comprises a rotor 320 magnetically supported in a housing 301 and rotatable about a rotation axis X for conveying a fluid, in particular blood, from an inlet 302 to an outlet 303, and a stator 340 formed in the housing 301.

In the example shown, the inlet 302 is connectable directly (by implantation in a heart wall) to a ventricle of a heart; the outlet 303 is connectable by means of a cannula to a blood vessel connected to the heart. The blood pump may therefore be used as a VAD.

The stator 340 comprises a motor coil arrangement 343, the rotor 320 and a motor magnet arrangement 324, so that a variable stator magnetic field may be generated by energizing the motor coils, by means of which the rotor 320 may be set in rotation.

The stator 340 is configured to generate the variable stator magnetic field in such a way that a bearing force that may be varied in accordance with a bearing control signal acts on the rotor 320 along a bearing direction of action. The rotor 320 is therefore actively magnetically supported in the housing 301 along the bearing direction of action by means of the variable bearing force. The motor coil arrangement 343 and the motor magnet arrangement 324 act as bearing magnets. Additionally or alternatively, the stator 340 may comprise at least one optional control coil 344 which acts to exert the bearing force on at least one magnet of the rotor 320, in the example shown on the inlet-side rotor magnet bearing 322. The bearing control signal corresponds to a voltage to be applied to the motor coil arrangement 343 and/or the control coil 344.

The bearing direction of action is substantially parallel to the rotation axis X and is also referred to below as the axial direction. Perpendicular directions are therefore referred to as radial or lateral directions. The bearing direction of action may be arranged in a direction other than the axial direction, for example in the radial or lateral direction or in a direction at a different angle to the rotation axis X.

In operation, the fluid enters the housing 301 and the rotor 320 substantially in the axial direction and is conveyed by a blading 321 in the radial direction into a volute 304 of the housing 301, from where it reaches the outlet 303. The blood flow is schematically illustrated by arrows in FIG. 1.

The blood pump 300 of the example shown is therefore embodied as a radial or centrifugal pump. However, the subject matter of the application is not limited to this type of pump, but is applicable to various types of rotary fluid pumps. For example, the blood pump may be an axial pump.

The rotor 320 is magnetically supported in the housing 301 in all spatial directions. The rotor 320 has passive magnetic support in the radial direction. To this end, the rotor 320 has an inlet-side rotor magnet bearing 322 and an outlet-side rotor magnet bearing 323, and the stator 340 has an inlet-side stator magnet bearing 341 and an outlet-side stator magnet bearing 342, with the inlet-side stator magnet bearing 341 acting on the inlet-side rotor magnet bearing 322 and the outlet-side stator magnet bearing 342 acting on the outlet-side rotor magnet bearing 323. As described above, the rotor 321 has an active magnetic support in the axial direction. However, the subject matter of the application is not limited to the bearing geometry described, but may be applied to various pumps with active magnetic support along at least one bearing direction of action.

The blood pump 300 is configured to provide a measurement signal dependent on a bearing position of the rotor 320 along the bearing direction of action. The control unit 200 is connected to the blood pump 300 by means of a driveline 305 for transmitting the measurement signal and the bearing control signal.

The measurement signal corresponds to a back-electromotive force (back-EMF) induced due to a rotation of the rotor 320, which may be detected as a voltage induced in the motor coil arrangement 343, which therefore corresponds to a “sensorless” generation of the measurement signal. The motor magnet arrangement 324 and/or the rotor magnet bearing 323 on the inlet side act as a sensor target arranged eccentrically in relation to the rotation axis X. The blood pump may alternatively or additionally comprise a sensor, for example an eddy current sensor and/or a magnetic field sensor, in particular a Hall sensor, to provide the measurement signal.

The measurement signal comprises a disturbance component dependent on a rotation of the rotor 320 about the rotation axis X and any lateral movement of the rotor perpendicular to the bearing direction of action, which in the case described results from the fact that the back-EMF depends not only on the bearing position of the rotor 320 along the bearing direction of action, but also on the rotation and lateral movement of the rotor 320, wherein such lateral movement may in particular periodically accompany the rotation.

A time course of the disturbance component is therefore substantially periodic with the rotation period of the rotor 320, but may also include a slowly varying component compared to the rotation period and/or may also be speed-dependent. Periodic components with periods of more than one rotation period of the rotor 320 may also occur.

FIG. 2 shows examples of various measured time-dependent position signals 401, 402, 403, which were determined on the basis of various measurement signals, each of which depends on the bearing position of a rotor in a blood pump of the type described along the bearing direction of action. The position signal 401 is based on a measurement signal corresponding to a back-EMF, as explained in the above description of the example shown in FIG. 1. The position signal 402 is based on a measurement signal from an eddy current sensor. The position signal 403 is based on a measurement signal from a Hall sensor. It is clearly recognizable that each of the position signals 401, 402, 403 comprises a component with a periodic time course. This component is caused by the rotation of the rotor and any lateral movement of the rotor associated with this rotation and is superimposed on the bearing position of the rotor relevant for controlling the active magnetic support. The component with a periodic time course is therefore a disturbance component in the above sense. This does not depend, or only insignificantly depends, on the bearing position of the rotor along the bearing direction of action averaged over a rotation period.

The control unit 200 shown in FIG. 1 is configured to detect the measurement signal and to determine a disturbance signal dependent on the disturbance component on the basis of the measurement signal detected in a detection interval. The detection interval comprises at least one, preferably a plurality of, rotation periods. The disturbance signal is determined in such a way that it is substantially periodic with the rotation period.

Furthermore, the control unit 200 is configured to determine, on the basis of the measurement signal and the determined disturbance signal, a specification for the bearing control signal in such a way that the rotor 320 is supported in the housing 301 without contact by means of the bearing force, wherein a power consumed for exerting the bearing force is reduced compared with a specification for the bearing control signal which may be determined without taking the disturbance signal into account.

The control unit 200 is furthermore configured to control the stator 320 according to the bearing control signal in accordance with the determined specification, wherein the control takes place after the detection interval. The control unit 200 is configured to carry out the detection of the measurement signal, the determination of the disturbance signal, the determination of the specification for the bearing control signal and the corresponding actuation of the stator 340 several times repeatedly in a sequence of control cycles.

The control unit 200 is configured to determine the disturbance signal by means of a learning control, as explained in more detail below in various examples of pump controls with reference to FIG. 3 to FIG. 4c.

Various learning control methods, in particular iteratively learning control, repetitive control or run-to-run control, may be used to determine the disturbance signal. Since such control methods are advantageously based on a stable system, in the example shown in FIG. 1 it is intended to stabilize the controlled system formed by stator 340 and rotor 320 by means of a bearing control. This is because the controlled system formed by stator 340 and rotor 320 is unstable with regard to control of the bearing position of the rotor 320 along the bearing direction of action.

As shown schematically in FIG. 3, the control unit 320 is therefore configured to stabilize this unstable controlled system in a closed control loop by means of a position controller, whereby the stabilized controlled system 501 is formed. Regardless of which manipulated and controlled variables are actually selected for the bearing control—various options are possible here, as explained below—the components of the manipulated variable vector uᵢ represent the manipulated variable for a number of time steps of a given control cycle i, the components of the manipulated variable vector uᵢ₊₁ represent the manipulated variable for a plurality of time steps of the subsequent control cycle i+1, the components of the controlled variable vector yᵢ represent the controlled variable for the plurality of time steps of control cycle i. The stabilized controlled system 501 is subjected to the manipulated variable vector uᵢ. In addition, the manipulated variable vector uᵢ and the controlled variable vector yᵢ are stored in a memory 502 of the control unit 320 and fed to a learning control 503, in the example described an iteratively learning control. On the basis of the manipulated variable vector u; and on the basis of the controlled variable vector yᵢ and on the basis of a periodic reference vector r, the iteratively learning control 503 determines the manipulated variable vector uᵢ₊₁ for the following control cycle. The periodic reference vector r may be set to zero, for example, if a corresponding control target is to be achieved. In the following control cycle, the manipulated variable vector uᵢ₊₁ replaces the manipulated variable vector uᵢ.

A model of the controlled system is required for the configuration of the learning control (see equations 1 to 3) in order to be able to determine specifications for correcting the control error. The complex dynamic behavior of the unstable (stabilized) controlled system 501 must be taken into account here; this is taken into account, for example, in the iterative learning control in the form of a quadratic learning matrix used here, with which a control error vector calculated as (r−yᵢ) is multiplied in each control cycle. Examples of procedures for the interpretation of iterative learning controls in general are given in the following publication and are therefore not repeated here in detail: Douglas A. Bristow, Marina Tharayil, Andrew G. Alleyne, Survey Of Iterative Learning Control: A Learning-Based Method for High-Performance Tracking Control, IEEE Control Systems, Volume 26, Issue 3, Pages 96-114, 2006, ISSN 1066-033X, doi: 10.1109/MCS.2006.1636313.

The control unit 200 may further be configured to perform a start of the blood pump, i.e. a ramp-up of the rotation of the rotor 320 from standstill to a target rotor speed, using the learning control by first operating the rotor at a start rotor speed that is lower than the target rotor speed and then repeating the following steps until the target rotor speed is reached: determining the disturbance signal by means of the learning control and disturbance compensation as described below (in conjunction with FIGS. 4a to 4c), increasing the rotor speed by one speed increment.

Assuming a linear transfer function of the bearing control from the measured variable (in particular the bearing position) to the manipulated variable (in particular the bearing force), the learning control may be carried out in the spectral range, i.e. the control unit 200 may be configured to split the disturbance signal into a plurality of frequency components and to carry out the disturbance compensation for each of the frequency components independently. The control unit 200 may also be configured to optimize a compensation variable for at least one—in particular the largest-, a plurality of, or all frequency components of the disturbance signal by means of an optimization method, for example the Newtonian method or the gradient descent method, and/or to adjust it using a transfer function of the position control in the spectral range.

Various combinations of manipulated and controlled variables may be provided for the learning control. Examples of corresponding pump controls are shown in FIG. 4a to FIG. 4c. In these examples, the learning control 508 may be an iteratively learning control or a learning control of another type, in particular a repetitive control or a run-to-run control.

In the pump control shown in FIG. 4a, it is provided that a controlled variable of the learning control is a target bearing force and a manipulated variable of the learning control is the bearing position of the rotor 320. The controlled system for the learning control 508 is the transfer function G_{lc} (s) of the closed control loop, comprising the bearing control 504 and the system consisting of stator 340 and rotor 320, which may be represented as

$${G}_{lc} ( s ) = - C ( s ) / ( 1 + G ( s ) C ( s ) ) , \qquad ( equation 1 )$$

wherein C (s) is a transfer function of the position controller 504 and G (s) is a transfer function of the controlled system consisting of stator 340 and rotor 320. The controlled system consisting of stator 340 and rotor 320 is shown in FIGS. 4a to 4c broken down into the control components actuator 505 and pump process element 506.

In the example shown in FIG. 4a, the learning control 508 determines an estimated sensor disturbance 507 as a disturbance signal. Here, the measurement signal output by the pump process element 506 is an uncorrected measurement signal and the control unit 200 is configured to determine a corrected measurement signal by applying the determined disturbance signal as a compensation signal to the measurement signal (referred to as disturbance compensation in the drawing), for example by adding the inverse disturbance signal, when determining the bearing control signal. Thus, a disturbance component of the corrected measurement signal that depends on the rotation of the rotor 320 about the rotation axis X or the lateral movement of the rotor 320 perpendicular to the rotation axis X is lower than the disturbance component of the uncorrected measurement signal. Block 507 is optional. It is also possible to add the output of the learning control 508 directly to the uncorrected measurement signal.

In the pump control shown in FIG. 4b, it is intended that a controlled variable of the learning control is a target bearing force and a manipulated variable of the learning control is the bearing force exerted on the rotor 320. In this case, a representation of the transfer function G_{lc} (s) of the closed control loop is given by

$${G}_{lc} ( s ) = 1 / ( 1 + G ( s ) C ( s ) ) . \qquad ( equation 2 )$$

In this example, the control unit 200 is configured to determine the bearing control signal in such a way that the rotor 320 is adjusted to a target bearing position at which external forces acting on the rotor 320 along the bearing direction of action add up to a predetermined force, in particular a zero force.

The control unit 200 is configured to determine the bearing control signal as a corrected bearing control signal, wherein in this case, during disturbance compensation, the disturbance signal determined by the learning controller 508 is applied as a compensation signal to an uncorrected bearing control signal (disturbed target bearing force output by the bearing controller 504) determined on the basis of the measurement signal. When the stator 340 is controlled in accordance with the corrected bearing control signal, the bearing force exerted on the rotor 320 is therefore less influenced by the disturbance component of the measurement signal than when the stator 340 is controlled in accordance with the uncorrected bearing control signal.

In the pump control shown in FIG. 4c, it is provided that a controlled variable of the learning control is a control error of the bearing control and a manipulated variable of the learning control is the bearing position of the rotor 320 along the bearing direction of action. In this case, a representation of the transfer function G_{lc} (s) of the closed control loop is given by

$${G}_{ls} ( s ) = - 1 / ( 1 + G ( s ) C ( s ) ) . \qquad ( equation 3 )$$

As in the example according to FIG. 4a, in the example according to FIG. 4b the learning control also determines an estimated sensor disturbance 507 as a disturbance signal. Here, the measurement signal output by the pump process element 506 is again an uncorrected measurement signal and the control unit 200 is configured to determine a corrected measurement signal by applying the determined disturbance signal as a compensation signal to the measurement signal (referred to as disturbance compensation in the drawing) when determining the bearing control signal. Thus, a disturbance component of the corrected measurement signal that depends on the rotation of the rotor 320 about the rotation axis X or the lateral movement of the rotor 320 perpendicular to the rotation axis X is lower than the disturbance component of the uncorrected measurement signal.

Optionally, a measured and/or calculated and/or estimated rotation angle of the rotor 320 about the rotation axis X (hereinafter also referred to as the rotor angle) may be provided to the learning control 508. This is advantageous, for example, in order to achieve good control results even if the duration of the rotation period (period duration) varies.

This extension is shown in FIG. 4d as an example for the structure in FIG. 4a; the examples in FIG. 4b and FIG. 4c may be extended accordingly. The start and end of a period are determined from the rotor angle. If the period duration changes, the learning control 508 must be adapted for this new period duration. The previously determined disturbance signal may be scaled in time to the new period duration, for example using an interpolation method, so that what has been learned so far may continue to be used. For the example of the iteratively learning control described above using FIG. 3, this would mean that the manipulated variable vector uᵢ is scaled in time to the new period duration and then the new manipulated variable vector uᵢ₊₁ is calculated with the control adapted to the new period duration.

With the information of the rotor angle, it is possible to plot the determined disturbance signal 507 over the rotor angle. One advantage over storing the time curve is that the measurement signal is correctly corrected for the disturbance component dependent on the rotation of the rotor 320 about the rotation point X, even if the speed changes during a period.

In particular, it is possible to use the disturbance signal 507 for correction in every period, but to perform an adjustment of the disturbance signal 507 by the learning control 508 only in selected periods, for example only in periods in which the speed change was very small.

If the system behaves differently than expected, for example due to unmodeled effects, the learning control may also lead to increased power consumption. As a safety measure, a difference between a power consumption when using the disturbance-compensated specification and a power consumption when using the non-disturbance-compensated specification and/or a disturbance-compensated specification of a previous control cycle may be determined and selected depending on a certain difference between the disturbance-compensated specification and the non-disturbance-compensated specification and/or the disturbance-compensated specification of a previous control cycle.

For example, the current power consumption may be measured for one or more periods when the learning control is switched off. The learning control may then be applied for one or more periods and the power may then be measured again over one or more periods. If the power has increased, the adjustment of the disturbance signal made between the two measurements by the learning control is reversed or no longer applied. This may be done, for example, by copying the disturbance signal 507 before the first power measurement and applying the copied disturbance signal again if the power increases.

As may be inferred from the foregoing, the control unit 200 according to the examples described above is particularly suitable for carrying out a method for controlling the blood pump 300, wherein the method comprises:
detecting the measurement signal,
determining the disturbance signal, dependent on the disturbance component, on the basis of the measurement signal detected in the detection interval,
determining, on the basis of the measurement signal and the determined disturbance signal, a specification for the bearing control signal in such a way that the rotor 320 is supported in the housing 301 without contact by means of the bearing force, wherein a power consumed for exerting the bearing force is reduced compared with a specification for the bearing control signal which may be determined without taking the disturbance signal into account, and
after the detection interval, controlling the stator 340 according to the bearing control signal in accordance with the determined specification.

Certain aspects of a use of the control unit 200 and/or the pump system 100 and/or the method described are highlighted in the following with reference to FIG. 5 to FIG. 6b using the example of the iteratively learning control.

FIG. 5 shows measured time-dependent target bearing force signals 601, 602, wherein the target bearing force is a manipulated variable of the bearing control. The time-dependent target bearing force signal 601 corresponds to a specification for the bearing control signal determined on the basis of the measurement signal without taking the disturbance signal into account, the time-dependent target bearing force signal 602 corresponds to a specification for the bearing force signal determined on the basis of the measurement signal and the disturbance signal, wherein the latter has been determined using an iteratively learning control as described above.

FIG. 5 shows that the target bearing force corresponding to the target bearing force signal 602 comprises fluctuations of lower amplitude than the target bearing force corresponding to the target bearing force signal 601. In both cases, the rotor 320 was supported contact-free along the bearing direction of action in the housing 301 over the course of the measurement using the bearing control. Accordingly, the power consumed to exert the bearing force, in particular the power averaged over several rotation periods, when the stator 340 is controlled in accordance with the bearing control signal corresponding to the specification determined taking into account the disturbance signal is lower than a power that is consumed when the stator 340 is controlled in accordance with a bearing control signal that corresponds to a specification that may be determined on the basis of the measurement signal but without determining or taking into account the disturbance signal.

Further effects of the learning control are visible in FIG. 6a and FIG. 6b. FIG. 6a shows power consumption curves 603, 604, which represent the power consumed by the blood pump 300 as a function of a rotor speed of the rotor 320. Analogously to the situation described in conjunction with FIG. 5, the power consumption curve 603 shows the power consumed when the specification for the bearing control signal is determined without taking the disturbance signal into account, and the power consumption curve 604 shows the power consumed when the specification for the bearing control signal is determined taking the disturbance signal into account using an iteratively learning control.

FIG. 6b shows control force curves 605, 606, which represent a quadratic mean of the bearing force required for the active magnetic support of the rotor 320 as a function of the rotor speed of the rotor 320. Analogously to the situation described in conjunction with FIG. 5, the control force curve 605 shows the required bearing force when determining the specification for the bearing control signal without taking the disturbance signal into account, and the control force curve 606 shows the required bearing force when the specification for the bearing control signal is determined taking the disturbance signal into account using an iteratively learning control.

The application also relates to the following aspects:
1. A control unit (200) for a blood pump (300),
wherein the blood pump (300) comprises a rotor (320) magnetically supported in a housing (301) and rotatable about a rotation axis (X) for conveying a fluid, and a stator (340), wherein the stator (340) is configured to generate a variable stator magnetic field for exerting a bearing force on the rotor (320) which may be varied along a bearing direction of action in accordance with a bearing control signal;
wherein the blood pump (300) is configured to provide a measurement signal which is dependent on a bearing position of the rotor (320) along the bearing direction of action and which comprises a disturbance component dependent on a rotation of the rotor (320) about the rotation axis (X) and/or a lateral movement of the rotor (320) perpendicular to the bearing direction of action;
wherein the control unit (200) is configured
   to detect the measurement signal and determine a disturbance signal dependent on the disturbance component on the basis of the measurement signal detected in a detection interval,
   to determine, on the basis of the measurement signal and the determined disturbance signal, a specification for the bearing control signal in such a way that the rotor (320) is supported in the housing (301) without contact by means of the bearing force, wherein a power consumed for exerting the bearing force is reduced compared with a specification for the bearing control signal which may be determined without taking the disturbance signal into account, and
   to control the stator (340) according to the bearing control signal in accordance with the determined specification, wherein the control takes place after the detection interval.
2. The control unit (200) according to aspect 1, wherein a time course of the disturbance component and/or the disturbance signal is substantially periodic with a period of a rotation of the rotor (320).
3. The control unit (200) according to any one of the preceding aspects, wherein the disturbance component and/or the disturbance signal does not depend on the bearing position of the rotor (320) averaged over a period of a rotation of the rotor (320) along the bearing direction of action.
4. The control unit (200) according to any one of the preceding aspects, wherein the detection interval comprises at least one period of the rotation of the rotor (320), and/or wherein the power consumed for exerting the bearing force, which is reduced with respect to a predetermined value for the bearing control signal that may be determined without taking the disturbance signal into account, is a time-averaged power, in particular averaged over at least one period of the rotation of the rotor (320).
5. The control unit (200) according to any one of the preceding aspects, wherein the stator (340) and the rotor (320) form an unstable controlled system with respect to a control of the bearing position of the rotor (320) along the bearing direction of action.
6. The control unit (200) according to aspect 5, wherein the control unit (200) is configured to stabilize the unstable controlled system by means of a position controller.
7. Control unit (200) according to one of the preceding aspects, arranged to determine the disturbance signal by means of a learning control (503, 508), in particular an iterative learning control (503, 508) and/or a repetitive control and/or a run-to-run control.
8. The control unit (200) according to aspect 7,
wherein a controlled variable of the learning control (503, 508) is a target bearing force and a manipulated variable of the learning control (503, 508) is the bearing position of the rotor (320),
and/or wherein a controlled variable of the learning control (503, 508) is a target bearing force and a manipulated variable of the learning control (503, 508) is the bearing force exerted on the rotor (320),
and/or wherein a controlled variable of the learning control (503, 508) is a control error of the bearing controller and a manipulated variable of the learning control (503, 508) is the bearing position of the rotor (320) along the bearing direction of action.
9. The control unit (200) according to any one of the preceding aspects, configured to determine the bearing control signal as a corrected bearing control signal, wherein the determined disturbance signal is applied as a compensation signal to an uncorrected bearing control signal determined on the basis of the measurement signal, so that when the stator (340) is controlled in accordance with the corrected bearing control signal, the bearing force exerted on the rotor (320) is influenced less by the disturbance component of the measurement signal than when the stator (340) is controlled in accordance with the uncorrected bearing control signal.
10. The control unit (200) according to any one of the preceding aspects, wherein the measurement signal is an uncorrected measurement signal and the control unit (200) is configured to determine a corrected measurement signal by applying the determined disturbance signal as a compensation signal to the measurement signal when determining the bearing control signal, so that a disturbance component of the corrected measurement signal which depends on the rotation of the rotor (320) about the rotation axis (X) or the lateral movement of the rotor (320) perpendicular to the rotation axis (X) is less than the disturbance component of the uncorrected measurement signal.
11. The control unit (200) according to any one of the preceding aspects, wherein the control unit (200) is configured to determine the bearing control signal in such a way that the rotor (320) is adjusted into a target bearing position at which external forces acting on the rotor (320) along the bearing direction of action add up to a predetermined force, in particular a zero force, and/or at which a power applied to generate the variable stator magnetic field is minimal.
12. A pump system (100) comprising
   a blood pump (300),
   wherein the blood pump (300) comprises a rotor (320) magnetically supported in a housing (301) and rotatable about a rotation axis (X) for conveying a fluid, and a stator (340), wherein the stator (340) is configured to generate a variable stator magnetic field for exerting a bearing force on the rotor (320) which may be varied along a bearing direction of action in accordance with a bearing control signal;
   wherein the blood pump (300) is configured to provide a measurement signal which is dependent on a bearing position of the rotor (320) along the bearing direction of action and which comprises a disturbance component dependent on a rotation of the rotor (320) about the rotation axis (X) and/or a lateral movement of the rotor (320) perpendicular to the bearing direction of action;
a control unit (200) according to any one of the preceding aspects, configured to control the blood pump (300).
13. The pump system (100) according to aspect 12, wherein the rotor (320) comprises a sensor target which is arranged eccentrically and/or not rotationally symmetrical with respect to the rotation axis (X), and the blood pump (300) is configured to provide the measurement signal on the basis of a position of the sensor target.
14. The pump system (100) according to aspect 12 or 13, wherein the measurement signal corresponds to a back-electromotive force (Back-EMF) induced due to a rotation of the rotor (320), and/or wherein the blood pump (300) comprises a sensor, in particular a Hall sensor, for providing the measurement signal.
15. The pump system (100) according to any one of aspects 12 to 14, wherein the bearing direction of action is arranged substantially parallel to the rotation axis (X).
16. A method for controlling a blood pump (300),
wherein the blood pump (300) comprises a rotor (320) magnetically supported in a housing (301) and rotatable about a rotation axis (X) for conveying a fluid, and a stator (340), wherein the stator (340) is configured to generate a variable stator magnetic field for exerting a bearing force on the rotor (320) which may be varied along a bearing direction of action in accordance with a bearing control signal;
wherein the blood pump (300) is configured to provide a measurement signal which is dependent on a bearing position of the rotor (320) along the bearing direction of action and which comprises a disturbance component dependent on a rotation of the rotor (320) about the rotation axis (X) and/or a lateral movement of the rotor (320) perpendicular to the bearing direction of action;
wherein the method comprises:
   detecting the measurement signal,
   determining a disturbance signal, dependent on the disturbance component, on the basis of the measurement signal detected in a detection interval,
   determining, on the basis of the measurement signal and the determined disturbance signal, a specification for the bearing control signal in such a way that the rotor (320) is supported in the housing (301) without contact by means of the bearing force, wherein a power consumed for exerting the bearing force is reduced compared with a specification for the bearing control signal which may be determined without taking the disturbance signal into account, and
   after the detection interval, controlling the stator (340) according to the bearing control signal in accordance with the determined specification.

### LIST OF REFERENCE SIGNS

100 pump system,
200 control unit,
300 blood pump,
301 housing,
302 inlet,
303 outlet,
304 volute,
305 driveline,
320 rotor,
321 blading,
322 inlet-side rotor magnet bearing,
323 outlet-side rotor magnet bearing,
324 motor magnet arrangement,
340 stator,
341 inlet-side stator magnet bearing,
342 outlet-side stator magnet bearing,
343 motor coil arrangement,
344 control coil,
401, 402, 403 time-dependent position signal,
501 stabilized controlled system,
502 memory,
503 iteratively learning control,
504 bearing control,
505 actuator,
506 pump process element,
507 estimated sensor disturbance,
508 learning control,
601, 602 time-dependent target bearing force signal,
603, 604 power consumption curve,
605, 606 control force curve,
X rotation axis.

1. A control unit for a blood pump,
comprising a rotor magnetically supported in a housing and rotatable about a rotation axis for conveying a fluid, and a stator configured to generate a variable stator magnetic field for exerting a bearing force on the rotor which may be varied along a bearing direction of action in accordance with a bearing control signal;
wherein the blood pump is configured to provide a measurement signal which is dependent on a bearing position of the rotor along the bearing direction of action and which comprises a disturbance component dependent on at least one of a rotation of the rotor about the rotation axis and/or a lateral movement of the rotor perpendicular to the bearing direction of action;
wherein the control unit is configured
to detect the measurement signal and determine a disturbance signal dependent on the disturbance component on the basis of the measurement signal detected in a detection interval,
to determine, on the basis of the measurement signal and the determined disturbance signal, a specification for the bearing control signal in such a way that the rotor is supported in the housing without contact by means of the bearing force, wherein a power consumed for exerting the bearing force is reduced compared with a specification for the bearing control signal which may be determined without taking the disturbance signal into account, and
to control the stator according to the bearing control signal in accordance with the determined specification, wherein the control takes place after the detection interval.

2. The control unit of claim 1, wherein at least one of a time course of the disturbance component and the disturbance signal is substantially periodic with a period of a rotation of the rotor.

3. The control unit of claim 1, wherein at least one of the disturbance component and the disturbance signal does not depend on the bearing position of the rotor along the bearing direction of action averaged over a period of a rotation of the rotor.

4. The control unit of claim 1, wherein at least one of:
the detection interval comprises at least one period of the rotation of the rotor, and
power consumed for exerting the bearing force, which is reduced with respect to a predetermined value for the bearing control signal that may be determined without taking the disturbance signal into account, is a time-averaged power averaged over at least one period of the rotation of the rotor.

5. The control unit of claim 1, wherein the stator and the rotor form an unstable controlled system with respect to a control of the bearing position of the rotor along the bearing direction of action.

6. The control unit of claim 5, wherein the control unit is configured to stabilize the unstable controlled system by means of a position controller.

7. The control unit of claim 1, configured to determine the disturbance signal by means of a learning control, where the learning control is at least one of an iteratively learning control, a repetitive control, and a run-to-run control.

8. The control unit of claim 7, wherein at least one of:
a controlled variable of the learning control is a target bearing force and a manipulated variable of the learning control is the bearing position of the rotor,
a controlled variable of the learning control is a target bearing force and a manipulated variable of the learning control is the bearing force exerted on the rotor, and
a controlled variable of the learning control is a control error of the bearing controller and a manipulated variable of the learning control is the bearing position of the rotor along the bearing direction of action.

9. The control unit of claim 1, wherein the learning control is configured to determine a start and end time of a period of a rotation of the rotor on the basis of a rotation angle of the rotor about the rotation axis.

10. The control unit of claim 1, configured to store a course of the disturbance signal as a function of a rotation angle of the rotor about the rotation axis.

11. The control unit of claim 1, configured to determine the bearing control signal as a corrected bearing control signal, wherein the determined disturbance signal is applied as a compensation signal to an uncorrected bearing control signal determined on the basis of the measurement signal, so that when the stator is controlled in accordance with the corrected bearing control signal, the bearing force exerted on the rotor is influenced less by the disturbance component of the measurement signal than when the stator is controlled in accordance with the uncorrected bearing control signal.

12. The control unit of claim 1, wherein the measurement signal is an uncorrected measurement signal and the control unit is configured to determine a corrected measurement signal by applying the determined disturbance signal as a compensation signal to the measurement signal when determining the bearing control signal, so that a disturbance component of the corrected measurement signal which depends on the rotation of the rotor about the rotation axis or the lateral movement of the rotor perpendicular to the rotation axis is less than the disturbance component of the uncorrected measurement signal.

13. The control unit of claim 1, wherein the control unit is configured to determine the bearing control signal in such a way that the rotor is adjusted into a target bearing position at which external forces acting on the rotor along the bearing direction of action add up to a predetermined force, wherein the predetermined force is a zero force, and/or at which a power applied to generate the variable stator magnetic field is minimal.

14. The control unit of claim 1, configured
to determine a difference between a first power consumption corresponding to the power consumed for exerting the bearing force when using the specification for the bearing control signal determined on the basis of the measurement signal and the disturbance signal, and a second power consumption corresponding to the power consumed for exerting the bearing force when using the specification for the bearing control signal that may be determined without taking the disturbance signal into account,
and, if the first power consumption is greater than the second power consumption, to control the stator according to the bearing control signal in accordance with the specification that may be determined without taking the disturbance signal into account or according to the bearing control signal in accordance with the specification determined in a previous control cycle.

15. A pump system comprising
a blood pump;
comprising a rotor magnetically supported in a housing and rotatable about a rotation axis for conveying a fluid, and a stator configured to generate a variable stator magnetic field for exerting a bearing force on the rotor which may be varied along a bearing direction of action in accordance with a bearing control signal;
wherein the blood pump is configured to provide a measurement signal which is dependent on a bearing position of the rotor along the bearing direction of action and which comprises a disturbance component dependent on at least one of a rotation of the rotor about the rotation axis and a lateral movement of the rotor perpendicular to the bearing direction of action;
a control unit of claim 1, configured to control the blood pump.

16. The pump system of claim 15, wherein the rotor comprises a sensor target which is arranged eccentrically and/or not rotationally symmetrical with respect to the rotation axis, and the blood pump is configured to provide the measurement signal on the basis of a position of the sensor target.

17. The pump system of claim 15, wherein at least one of:
the measurement signal corresponds to a back-electromotive force induced due to a rotation of the rotor, and
the blood pump comprises a Hall sensor, for providing the measurement signal.

18. The pump system of claim 15, wherein the bearing direction of action is arranged substantially parallel to the rotation axis.

49. A method for controlling a blood pump
comprising a rotor magnetically supported in a housing and rotatable about a rotation axis for conveying a fluid, and a stator configured to generate a variable stator magnetic field for exerting a bearing force on the rotor which may be varied along a bearing direction of action in accordance with a bearing control signal;
wherein the blood pump is configured to provide a measurement signal which is dependent on a bearing position of the rotor along the bearing direction of action and which comprises a disturbance component dependent on at least one of a rotation of the rotor about the rotation axis and a lateral movement of the rotor perpendicular to the bearing direction of action;
wherein the method comprises:
detecting the measurement signal,
determining a disturbance signal, dependent on the disturbance component, on the basis of the measurement signal detected in a detection interval,
determining, on the basis of the measurement signal and the determined disturbance signal, a specification for the bearing control signal in such a way that the rotor is supported in the housing without contact by means of the bearing force, wherein a power consumed for exerting the bearing force is reduced compared with a specification for the bearing control signal which may be determined without taking the disturbance signal into account, and
after the detection interval, controlling the stator according to the bearing control signal in accordance with the determined specification.

20. The pump system of claim 16, wherein at least one of:
the measurement signal corresponds to a back-electromotive force induced due to a rotation of the rotor, and
the blood pump comprises a sensor, in particular a Hall sensor, for providing the measurement signal.
